(19) United States
(12) Patent Application Publication
(10) Pub. No.: US 2011/0160680 A1
(43) Pub. Date: Jun. 30, 2011

(54) WIRE GUIDE WITH CANNULA

(75) Inventors: Logan M. Cage, Bloomington, IN (US); Aaron Weeks, Bloomington, IN (US); Thomas A. Kay, JR., Bloomington, IN (US); James C. Elsesser, Bloomington, IN (US)

(73) Assignee: Cook Incorporated, Bloomington, IN (US)

(21) Appl. No.: 12/648,724

(22) Filed: Dec. 29, 2009

(51) Int. Cl. A61M 25/09; A61L 15/50; B23P 17/04

(52) U.S. Cl. 604/265; 604/528; 29/428

(57) ABSTRACT

The present invention generally relates to a medical surgical device and specifically a wire guide for percutaneous placement within a body lumen. The wire guide includes an elongated mandrel having sufficient flexibility and kink resistance and a cannula having a stiffness greater than the mandrel stiffness for sufficient pushability. The mandrel extends through the cannula and has a portion extending distal thereto to form a distal tip of the wire guide. The wire guide can further include a slotted cannula to improve the flexibility of the wire guide at the proximal portion of the wire guide. The slots can be arranged in various patterns for increased flexibility along the longitudinal length of the cannula, as well as increased circumferential flexibility along the wire guide. The mandrel may also have a proximally tapered portion to improve the flexibility of the wire guide at the proximal portion of the wire guide.

### TECHNICAL FIELD

The present invention generally relates to a medical surgical device and specifically a wire guide for percutaneous placement within a body lumen. The flexibility of the wire guide can be varied by a cannula coaxially displaced on the wire core.

### BACKGROUND

Wire guides are commonly used in vascular procedures, such as angioplasty procedures, diagnostic and interventional procedures, percutaneous access procedures, or radiological and neuroradiological procedures in general, to introduce a wide variety of medical devices into the vascular system. For example, wire guides are used for advancing intraluminal devices such as stent delivery catheters, balloon dilation catheters, atherectomy catheters, and the like within body lumens. Typically, the wire guide is positioned inside the inner lumen of an introducer catheter. The wire guide is advanced out of the distal end of the introducer catheter into the patient until the distal end of the wire guide reaches the location where the interventional procedure is to be performed. After the wire guide is inserted, another device such as a stent and stent delivery catheter is advanced over the previously introduced wire guide into the patient until the stent delivery catheter is in the desired location. After the stent has been delivered, the stent delivery catheter can then be removed from a patient by retracting the stent delivery catheter back over the wire guide. The wire guide may be left in place after the procedure is completed to ensure easy access if an additional procedure is required.

Conventional wire guides include an elongated wire core with one or more tapered sections near the distal end to increase flexibility. Generally, a flexible body such as a helical coil or tubular body is disposed about the wire core. The wire core is secured to the flexible body at the distal end. Especially for small diameter wire guides (0.014-0.018 inches), the wire core is made of either stainless steel or Nitinol with each having their own desired properties of flexibility with sufficient kink resistance and stiffness. In addition, a torquing means can be provided on the proximal end of the core member to rotate, and thereby steer a wire guide having a curved tip, as it is being advanced through a patient's vascular system.

A major requirement for wire guides and other intraluminal guiding members is that they have sufficient stiffness to be pushed through the patient's vascular system or other body lumen without kinking. However, they must also be flexible enough to pass through the tortuous passageways without damaging the blood vessel or any other body lumen through which they are advanced. Efforts have been made to improve both the strength and the flexibility of wire guides to make them more suitable for their intended uses, but these two properties tend to be diametrically opposed to one another in that an increase in one usually involves a decrease in the other.

For certain procedures, such as when delivering stents around challenging take-off, tortuosities, or severe angulation, substantially more support and/or vessel straightening is frequently needed from the wire guide. Wire guides that provide improved support over conventional wire guides have been commercially available for such procedures. However, such wire guides are in some instances so stiff they can damage vessel linings when being advanced.

For example, some wire guides comprise an assembly of a shape memory alloy wire core and cladding covering of a different metal extending along the wire core entirely. The assembly is drawn down in multiple steps to form an intimate mechanical bond between the wire core and cladded covering, thereby forming a wire of the composite structure. Annealing and cold working is typically required between drawing passes in order to achieve the desired properties of the composite. A combination of drawing and heat-treating and/or pressure-treating a cladded assembly is difficult and expensive, especially the manufacturing intensive steps to draw down fully to a wire guide size of about 0.018 inches. There is also excessive wear on the drawing equipment due to the abrasiveness of the shape memory core. The drawn composite structure usually requires the cladding covering material and the core material to run the entire length of the composite. This is problematic when the core wire alone has the desired flexibility, and the cladding covering material increases undesirably the stiffness of the distal end of the wire core.

In view of the above, it is apparent that there exists a need for an improved design for a wire guide.

### BRIEF SUMMARY

One aspect provides a wire guide having sufficient flexibility and kink resistance, as well as sufficient pushability. In one embodiment, the wire guide includes an elongated mandrel and a cannula having a stiffness greater than the mandrel stiffness. The mandrel can be made of a shape memory alloy such as Nitinol and the cannula can be made of a metal alloy such as stainless steel, platinum, palladium, a nickel-titanium, or combinations thereof. The cannula lumen is sized to receive the mandrel therein, and has a length relative to the length of the mandrel such that a portion of the mandrel extends distal to the cannula when received in the cannula lumen. The extended distal portion of the mandrel can permit the wire guide to have sufficient flexibility and kink resistance at its distal region, while the cannula can permit the wire guide to have sufficient pushability at its proximal region. The cannula can be attached, preferably by soldering or welding, along the length of the mandrel at least at one attachment joint. The extended distal portion of the mandrel can have a taper from a first diameter to a second diameter to form a distal tip of the wire guide. A proximal portion of the mandrel may also have a taper from a first diameter to a second diameter to improve the flexibility along the proximal region of the wire guide. The wire guide may also include a polymer coating surrounding the extended portion of the mandrel to further form the distal tip, a hydrophilic coating disposed on a portion of the polymer coating, a lubricious polymer coating disposed on at least one of a portion of the cannula or a portion of the extended portion of the wire guide, or any combination of coatings.

In yet another embodiment, the wire guide can further include a slotted cannula to improve the flexibility of the wire guide where the cannula is located. The slots are formed in the wall of the cannula and are circumferentially disposed along the cannula wall. The slots formed in the cannula wall can be transverse to the longitudinal axis of the wire guide. More than one slot can be formed along a single circumferential region such that a first slot is circumferentially spaced from a second slot. More than one series of slots can be formed in the wall of the cannula to vary the flexibility along the wire guide. The slot of a series can be spaced from an adjacent slot of the same series at a longitudinal distance. This longitudinal distance at a proximal portion of the cannula can be greater than the longitudinal distance at a distal portion of the cannula to increase the longitudinal flexibility along the wire guide. The slots of a series can be circumferentially positioned relative to one another at an angle to form a generally helical pattern along the cannula wall to vary the circumferential flexibility along the wire guide. Slots of a second series can be interposed between adjacent slots of a first series. The second series slots may be circumferentially positioned relative to each of the first series slots at an angle such that the second series slots are circumferentially offset from the first series.

Another aspect provides a method of making a wire guide having sufficient flexibility and kink resistance, as well as sufficient pushability for use within a body vessel. An elongated mandrel having a taper from a first diameter to a second diameter and a cannula having a lumen is provided. The cannula lumen is sized to receive the mandrel therein. The cannula has a stiffness greater than the mandrel stiffness. The elongated mandrel is inserted into the cannula lumen such that a portion of the mandrel extends distal to the cannula. The cannula is attached, preferably by soldering or welding, to the mandrel to form a wire guide. A plurality of slots can be formed in the wall of the cannula by laser cutting.

Other features of the present invention and the corresponding advantages of those features will become apparent from the following discussion of the preferred embodiments of the present invention, exemplifying the best mode of practicing the present invention, which is illustrated in the accompanying drawings. The components in the figures are not necessarily to scale, emphasis instead being placed upon illustrating the principles of the invention. Moreover, in the figures, like referenced numerals designate corresponding parts throughout the different views.

### BRIEF DESCRIPTION OF THE DRAWINGS

FIG. 1 is a side elevation view of a wire guide.

FIG. 2 is a side elevation view of a cannula with slots for use with a wire guide.

FIGS. 2A-2D are various transverse sectional views taken along different circumferential regions of the cannula of FIG. 2.

FIG. 3 is a side elevation view of a portion of a cannula with slots for use with a wire guide.

FIG. 3A is a transverse sectional view taken along a circumferential region of the cannula of FIG. 3.

FIG. 4 is a side elevation view of a wire guide, depicting a reverse tapered mandrel within the proximal portion.

### DETAILED DESCRIPTION

In accordance with an embodiment of the present invention, a wire guide system includes a wire guide having sufficient flexibility and kink resistance, as well as sufficient pushability for use in a body vessel of a human or animal patient (“patient”).

The terms proximal and distal are used herein to refer to portions of a wire guide. As used herein, the term “distal” is defined as that portion of the wire guide closest to the end of the wire guide inserted into the patient's body lumen. The term “proximal” is defined as that portion of the wire guide closest to the end of the wire guide that is not inserted into the patient's body lumen. The terms distally and proximally are used herein to refer to directions along an axis joining the proximal and distal portions of the wire guide (“axial direction”). For example, proximal movement is movement towards the proximal portion of the wire guide. Distal movement is movement towards the distal portion of the wire guide.

The wire guide of the preferred embodiments includes a mandrel or wire core of a material having a desirable flexibility and kink resistance, which can run the entire length of the wire guide. The wire guide further includes a cannula of a stiffer material than the mandrel material coaxially disposed around the proximal portion of the mandrel to improve the stiffness more effectively along the proximal region of the wire guide for better pushability. Accordingly, this wire guide has sufficient stiffness to be pushed through the patient's vascular system or other body lumen without severe kinking. Also, the wire guide has an increased flexibility and kink resistance at the distal end for initial insertion around a challenging take-off and for navigation through the vasculature with tortuosities and/or severe angulation without damaging the blood vessel or any other body lumen through which the wire guide is advanced through.

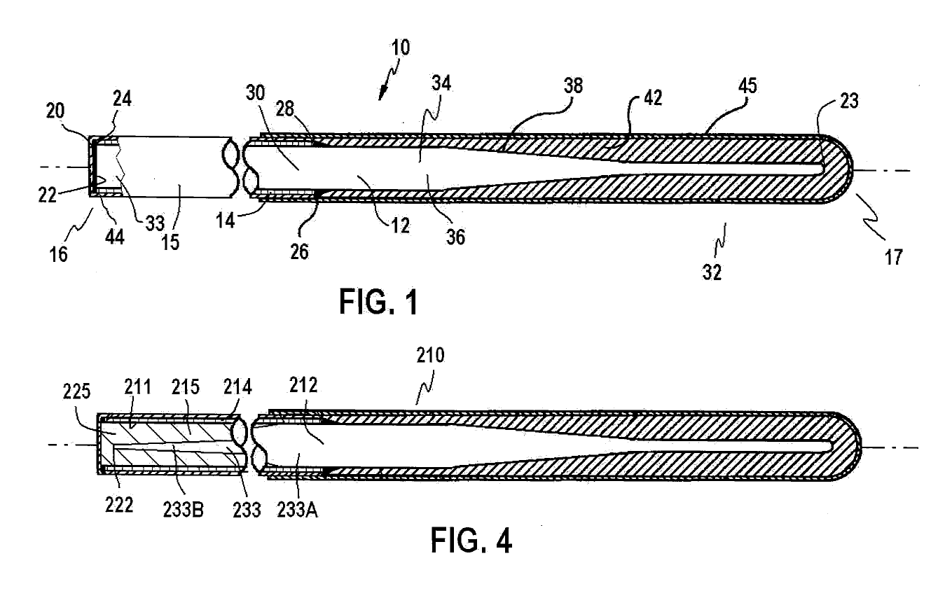

FIG. 1 is an illustration of a wire guide 10 in accordance with one of the preferred embodiments described herein. The longitudinal length of wire guide 10 may range from about 40 cm to about 480 cm, and the outside diameter of wire guide 10 may range from about 0.008 inches to about 0.05 inches, and preferably about 0.018 inches. The dimensions and configurations of various components described herein are particularly suitable for use in peripheral intervention, although the dimensions can vary as needed depending on the type of use in other applications, such as aortic intervention.

Wire guide 10 includes a body 15 having a mandrel or elongated central core 12 and a cannula 14. Mandrel 12 can be made from many materials having flexible, elastic or bendable properties in order to traverse the vessels or arteries with sufficient kink resistance. The mandrel material can include stainless steel, such as spring temper stainless steel, Nitinol, or other materials that have properties similar to stainless steel and Nitinol, e.g., properties of kink resistance, capability of withstanding sterilization (heat and moisture), and non-toxicity. Depending on the application, the mandrel can be made of stainless steel because of its preferred properties of pushability and stiffness, although stainless steel can be more likely to plastically deform or kink. Compared to stainless steel, the mandrel can be made of Nitinol because of its preferred properties of flexibility and kink resistance, although Nitinol is less desirable for stiffness and pushability. Preferably, the mandrel is made of Nitinol wire core of about 50/50 mix of Nickel and Titanium in a super-elastic condition at or below a room temperature of 0 degrees Celsius.

Cannula 14 is disposed coaxially around the mandrel 12, preferably defining a region from the proximal end 16 of wire guide 10 to an intermediate portion of the wire guide. The material of cannula 14 can be any material having a stiffness greater than the stiffness of the material of mandrel 12. For example, the cannula can be made of a metal alloy, such as stainless steel alloy, such as a hypotube catheter shaft, platinum alloy, palladium alloy, nickel-titanium alloy, or combinations thereof. Cannula 14 comprises a cylindrical tubular body having a lumen with an interior diameter that is sized to fit snugly over a substantial portion of mandrel 12. The interior diameter of the cannula lumen depends on the size of the mandrel and, e.g., can be in the range of about 0.008 inches to about 0.015 inches. The outer diameter of cannula 14 can be in the range of about 0.016 inches to about 0.02 inches. The longitudinal length of cannula 14 is shorter than the length of the mandrel, being any length to increase sufficiently the stiffness of the proximal portion of mandrel 12. Also, in alternative embodiments, a portion of cannula 14 may also have a rectangular shape or any other shape adapted to provide a good grip that helps the physician maneuver wire guide 10 through the vascular anatomy.

Cannula 14 can be attached to mandrel 12 at one or more joints by any numerous attachment methods, including adhesives, soldering, such as ultrasonic soldering, and welding, such as laser or fusion welding. For example, the embodiment shown in FIG. 1 has a first attachment joint 20 between the proximal end 22 of mandrel 12 and the proximal end 24 of cannula 14 and a second attachment joint 26 between the distal end 28 of the cannula and an intermediate portion 30 of the mandrel. Second attachment joint 26 is preferably circumferentially around the mandrel adjacent the distal end 28 of cannula 14. Second attachment joint 26 is shaped in order to form a smooth transition between the outer diameter of each of the cannula and the mandrel, respectively. The shaping of the second attachment joint is preferably accomplished by grinding and/or polishing. A suitable method for fusion welding titanium alloys to ferrous materials is taught by Edison Welding Institute of Columbus, Ohio in U.S. Pat. No. 6,875,949 to Hall, which is incorporated herein by reference in its entirety. Moreover, a skilled artisan is capable of selecting a suitable solder for joining a ferrous and non-ferrous material, such as a stainless steel cannula and a Nitinol mandrel. Suitable solders, such as SnAg (tin-silver) eutectic solder or AuSn (gold-tin) eutectic solder, for soldering are available from Indium Corporation of America of Utica, N.Y.

As shown in FIG. 1, mandrel 12 extends past the distal end of cannula 14 to define an extended portion 34 thereof. The region defined along extended portion 34 and the distal end 17 of wire guide 10 forms a distal tip 32 of wire guide 10. The distal tip of the wire guide without the cannula can be beneficial because the mandrel alone has the desired flexibility and kink resistance and is less traumatic to the body vessel. Although it is preferred to have one continuous mandrel throughout the wire guide, mandrel 12 may alternatively comprise of two or more axial components that are axially aligned and attached end-to-end to form the overall length of mandrel by methods known to skilled artisans.

In one embodiment, mandrel 12 has a substantial proximal portion 33 with a uniform outer diameter, typically associated with cannula 14. Proximal portion 33 of mandrel 12 has a longitudinal length that can range from about 20 cm to about 300 cm. To that end, the length of the cannula can be identical to the length of the proximal portion of the mandrel or much less, e.g., the length can be in the range of about 25% to about 100% the length of the mandrel, or about 100 cm to about 180 cm for a 300 cm mandrel. Extended portion 34 can be about 10 cm to about 30 cm.

Extended portion 34 of mandrel 14 can also include a portion 36 with the uniform outer diameter, as well as a distal tapering portion 38 which tapers from the uniform outer diameter to a smaller outer diameter, as shown in FIG. 1. The uniform outer diameter of extended portion 34 and/or proximal portion 33 can be in the range of about 0.007 inches to about 0.013 inches. Distal portion 38 of mandrel 12 tapers from the uniform outer diameter to a smaller outer diameter at the distal end 23 of the mandrel 12 at a rate of about 0.002 inches/1-inch span to about 0.004 inches/1-inch span. In one example, distal portion 38 of mandrel 12 tapers from an outer diameter of about 0.01 inches to an outer diameter of about 0.005 inches for a span of about 3 inches.

The entire length of the wire guide or discrete lengths of the wire guide can be coated with a coating. For example, wire guide 10, preferably distal tip 32, can be covered by a first coating 42 that sufficiently bonds to mandrel 14. First coating 42 can be a polymeric material, such as nylon, polyethylene, polyurethane, fluoropolymer or the like. The thickness of coating 42 will vary in order to give a substantially uniform diameter, removing the discontinuities along its length that might otherwise exist. The thickness of coating 42 can vary proportionally to the tapering rate to the mandrel, and can range from about 0.001 inches to about 0.01 inches. The distal portion of distal tip 32 may be shaped and configured to be atraumatic.

A substantial length of wire guide body 15, including cannula 14, may be covered by a second coating 44 that is in addition to or instead of first coating 42. Second coating 44 can be any polymeric material having a surface exhibiting a low coefficient of friction, preferably lower than the bare metal cannula. In preferred embodiments, coating 44 is polytetrafluoroethylene (Teflon), but can also be nylon, polyethylene, polyurethane, fluoropolymer or the like. The thickness of second coating 44 can range from about 0.00002 inches to about 0.08 inches, and preferably ranges from about 0.0002 inches to about 0.02 inches. When the cannula has slots as described later, the slots may also have a coating of material within to protect the body vessel from possible sharp edges of the slots and/or make the slotted cannula smoother along the entire length.

A third coating 45 may also be applied to the outside of first coating 42 and/or the outside of second coating 44. In some instances, first coating 42 acts as primer or adhesion promoter between the mandrel material and the hydrophilic coating. Third coating 45 preferably is a hydrophillic coating comprising polyvinylpryrrolidones, polyethylene oxides, polyacrylates or a mixture thereof. Preferably, third coating 45 forms an exterior surface of the distal portion of the wire guide, having a lubricity with a coefficient of friction at least as low as second coating 44, if not lower. Each of the coatings described herein can be applied by any method known to a skilled artisan, e.g., spray, extrusion, brush, dip, or any combination thereof. It is to be understood by one of ordinary skill in the art that the wire guide can be bare metal without any coating or can include various combinations of coatings as described herein.

In alternative embodiments, distal tip 32 may include a coil (not shown) disposed circumferentially around extended portion 34 of the mandrel 14. An example of a coil construction distal tip is found in U.S. Pat. No. 7,001,345 to Connors, III et al., which is incorporated herein by reference in its entirety.

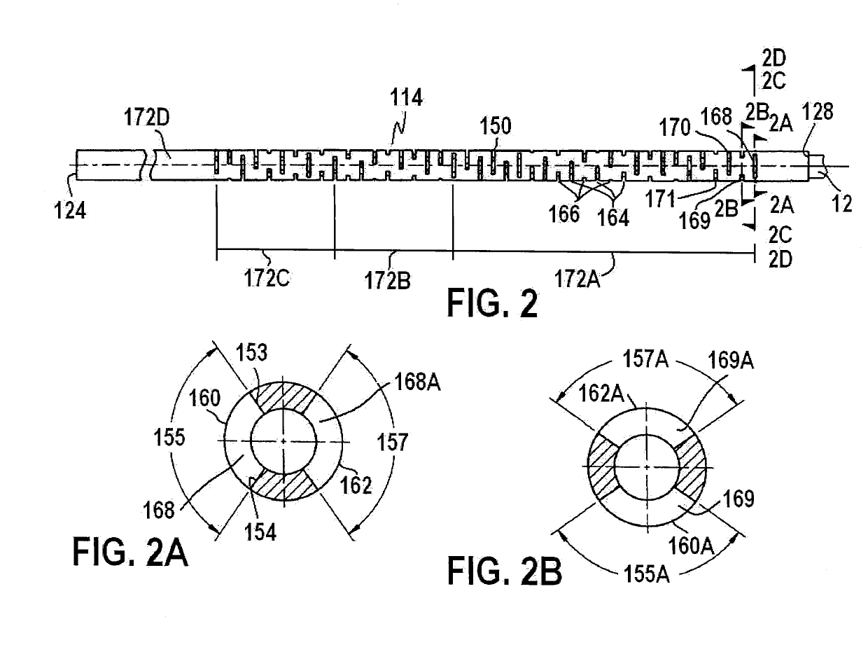

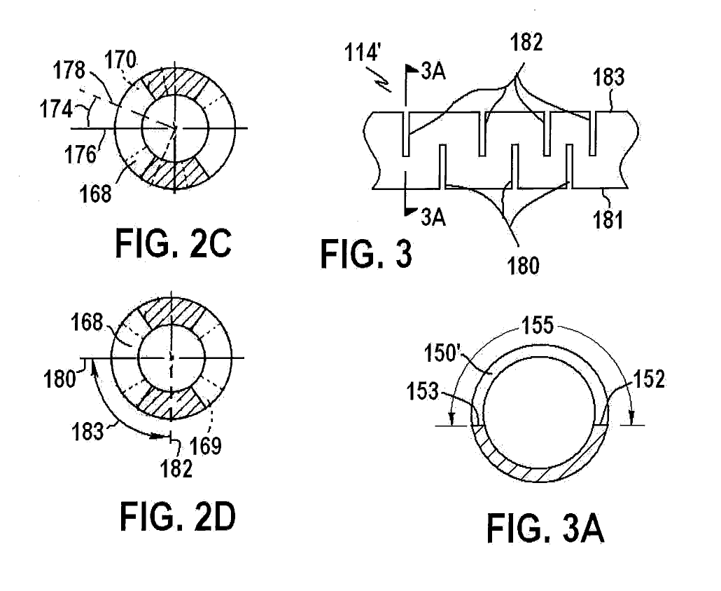

In some applications, it is desirable to have more flexibility toward the proximal end of the wire guide in order to distribute stress caused by flexing or bending the wire guide more evenly along the cannula, without sacrificing pushability performance. According to FIGS. 2-3A, cannula, referred to now as reference numeral 114, is substantially identical to cannula 14 except having one or more slots 150 sufficiently sized and oriented to improve flexibility of the wire guide, especially the proximal portion of the wire guide body associated with the cannula. Slots 150 can be formed in cannula 114 by any means known by a skilled artisan, but preferably the slots are formed in the cannula by laser cutting. The width or thickness of slot 150 can vary along the cannula depending on the application, and may be in the range of about 0.001 inches to about 0.002 inches. For example, the slot can have a uniform width of any distance along a portion of the cannula, and this uniform width may change along different portions of the cannula. Optionally, the cross-sectional width of the slot can be tapered instead of square which may improve the flexibility and structural integrity of the cannula.

The slots can have a variety of shapes and orientations. Preferably, slots 150 are formed to be transverse to the longitudinal axis into the wall of cannula 114. FIGS. 2A and 2B are transverse sectional views of the respective cannulas taken at a circumferential region of a particular location of the respective cannulas. According to FIG. 2A, the slot can be formed by removing a triangular portion having edges 153, 154 that are angled with respect to one another at any angle, e.g., between about 45 degrees to about 180 degrees. The slot can be arcuate or linearly formed, as shown in FIG. 3A, such that edges 152, 153 of the slot are parallel to a line tangential to the cannula, or at an angle of about 180 degrees from one another. The orientation and size of slot portions, as well as the cannula material adjacent the slot portions, can be characterized as angular segments with respect to 360 degree circumference of a region.

One slot may be formed along a portion of any circumferential region of the cannula, or optionally, two or more slots can be formed at a particular circumferential region of the cannula. For example, in FIG. 2A, a first slot 168 is disposed along a first portion 160 of a circumferential region at an angle 155 and a second slot 168A is disposed along a second portion 162 of the same circumferential region at an angle 157, which can be substantially identical to angle 155. First slot 168 can be positioned somewhere opposite to second slot 168A across the cannula lumen so that second slot 168A is circumferentially spaced from first slot 168 at different angles. Preferably, slots 168, 168A are positioned diametrically opposite as shown where second slot 168A is circumferentially spaced from first slot 168 at equal angles. When more than one slot is formed along the circumferential region, the circumferential angle between adjacent slots can vary or can be uniform.

Cannula 114 can have one or more series of slots. According to FIG. 2, cannula 114 includes a first series of slots 164 and a second series of slots 166. Each series of slots can be arranged identically with the same pattern or differently with individual patterns to obtain a desirable flexibility of the wire guide.

One factor for the arrangement of the slots is the longitudinal spacing between slots located at adjacent circumferential regions of the cannula. The longitudinal spacing between adjacent slots of the same series can be any distance depending on the application. For example, in FIG. 2 the longitudinal spacing between adjacent slots 168, 170 in a series can be in the range of about 0.008 inches or less to about 0.064 inches or more. The longitudinal spacing may vary along cannula 114, the longitudinal spacing may remain fixed along the cannula, or a combination of both. Preferably, the longitudinal spacing between adjacent slots toward the distal end 128 of cannula 114, e.g., slots 168, 170, is smaller than the longitudinal spacing between adjacent slots toward the proximal end 124 of cannula 114. Although depending on the application the longitudinal spacing between adjacent slots of a series can be instead larger along the same direction. In some examples, the longitudinal spacing between adjacent slots of a series can gradually decrease (or increase) in the distal direction along cannula 114, as shown in FIG. 3.

With reference to FIG. 2, the cannula slots can be further divided into longitudinal segments along cannula 114, e.g., a first, most distal, longitudinal slot 172A, a second longitudinal slot 172B adjacent the first segment, and a third longitudinal slot 172C adjacent the second segment. Each longitudinal segment can be of any length, although the length of the first longitudinal segment 172A is shown to be greater than each of the lengths of the second and third longitudinal segments 172B, 172C. Any number of longitudinal segments can be included along the cannula. The slots can end at some intermediate portion of the cannula such that a longitudinal segment of the cannula from the most proximal slot to the proximal end of the cannula is without any slots, where pushability and less flexibility are needed.

The number of longitudinal segments alone, or combined with the different arrangements of slots described herein, can vary the flexibility of the proximal portion of the wire guide at different rates. For example, the longitudinal spacing between adjacent slots of a series can be fixed within each of the longitudinal segments, but can also decrease (or increase) for each longitudinal segment in the distal direction along the cannula. According to FIG. 2, the longitudinal spacing between adjacent slots in a series is fixed along each of the first, second, and third longitudinal segment 172A-C.

Another factor for the arrangement of the slots is the relative angular placement between slots of a series located at adjacent circumferential regions of the cannula, which can be any angle depending on the application. FIG. 2B illustrates a circumferential region adjacent the circumferential region of FIG. 2A, having a first slot 169 disposed along a first portion 160A of a circumferential region at an angle 155A and a second slot 169A disposed along a second portion 162A of the same circumferential region at an angle 157A, which can be substantially identical to angle 155A. The slots of the circumferential region of FIG. 2B are also shown to be circumferentially offset about 90 degrees relative to the slots of circumferential region of FIG. 2A.

In FIG. 2, slot 168 of the first series 164 is disposed along a portion of a first circumferential region of the cannula 114, and an adjacent slot 170 of the same series is disposed along a portion of a second circumferential region of the cannula. FIG. 2C is a transverse sectional view of cannula 114 taken at a circumferential region, depicting the relative angular placement of adjacent slots within the same series of slots. According to FIG. 2C, the middle of slot 168 of the first circumferential region, represented by dashed line 176, and the middle of slot 170, shown as dashed lines, of the second circumferential region, represented by dashed line 178, can be circumferentially disposed relative to one another at an angle 174 between about 0 degrees and 90 degrees, and preferably about 1-30 degrees. FIG. 2C shows angle 174 of about 20 degrees. This relative angular placement between adjacent slots of a series forms an appearance of a generally helical arrangement of slots along the cannula, such that the wire guide can have a circumferential flexibility of 360 degrees without the operator having to rotate the shaft. Not only does the slot arrangement distribute the stress along the longitudinal length of the cannula, but also the slot arrangement distributes the stress circumferentially.

The slots as mentioned may include additional series of slots with the same arrangement as the first series or with different arrangements. The additional series can be associated with the entire cannula or only a longitudinal segment of the cannula. The additional series may be associated with the same longitudinal segments as the first series, or each series may be associated with different longitudinal segments so as to take advantage of the features of each pattern along different segments of the cannula. According to FIG. 2, each slot of second series 166 is interposed between adjacent slots of first series 164, preferably centrally located between the adjacent slots. As can be appreciated by one of ordinary skill in the art, the slots of the second series do not have to be disposed in consecutive adjacent first series slots, but can be located between every second, third, fourth, fifth, etc. adjacent first series slot for the desired flexibility. Also, a second, third, fourth, fifth series, etc. of slots can be included such that the second series slot is placed adjacent the first series, the third series is placed in the slot next to the second series slot, and so forth.

The longitudinal spacing between adjacent slots of different series can be any distance depending on the application. For example, the longitudinal spacing between slot 168 of the first series slots and slot 169 of the second series slots, adjacent to slot 168, can be in the range of about 0.008 inches or less to about 0.064 inches or more. Like the longitudinal spacing between adjacent slots in the same series, the longitudinal spacing between adjacent slots of different series can be fixed along the cannula, or can vary along the cannula. For example, the longitudinal spacing between adjacent slots of a different series can be fixed within each of the longitudinal segments, but can also decrease (or increase) for each longitudinal segment in the distal direction along the cannula. According to FIG. 2, the longitudinal spacing between adjacent slots in a different series is fixed along each of the first, second, and third longitudinal segment 172A-C.

The relative angular placement between adjacent slots of the first series 164 and the second series 166 can be any angle depending on the application. For example according to FIG. 2, slot 168 of the first series 164 is disposed along a portion of a circumferential region of cannula 114, and the adjacent slot 169 of the second series 166 is disposed along a portion of a circumferential region of the cannula. FIG. 2D is a transverse sectional view of cannula 114 taken at a circumferential region, depicting the relative angular placement of adjacent slots within a different series of slots. The middle of slot 168 of the first series 164, represented by a dashed line 180, and the middle of slot 169, shown as dashed lines, of the second series 166, represented by a dashed line 182, can be circumferentially disposed relative to one another at an angle 183 between about 20 degrees and 180 degrees. FIG. 2D shows an angle of about 90 degrees. This relative angular placement between slots of the first and second series forms an appearance of multiple helixes of slots along the cannula.

FIG. 3 illustrates a portion of another embodiment of a cannula with slots having a first series 180 of slots along the bottom 181 of the cannula 114′ and a second series 182 of slots along the top 183 of the cannula. Each slot of the second series 182 is interposed between adjacent slots of the first series 180, preferably centrally located between the adjacent first series slots. The slots may also extend more than 50% circumferentially around the cannula. FIG. 3A is a transverse sectional view of cannula 114′ taken at a circumferential region. In FIG. 3A, slot 150′ can be linearly formed, such that the edge of the slot is parallel to a line tangential to the cannula or that edges 152, 153 are at an angle of about 180 degrees from one another. Although one slot is shown, two or more slots can be formed, like, e.g., slot 150′, along the circumferential region. FIG. 3 also illustrates that the longitudinal spacing between adjacent slots of each series 180, 182 can gradually decrease (or increase) in the distal direction along cannula 114 to gradually increase the flexibility of the wire guide in the distal direction.

In accordance with FIG. 4, a wire guide 210 is identical to wire guide 10 except for the following. Proximal portion 233 of mandrel 212 has a distal region 233A with the uniform outer diameter, and a proximal tapering portion 233B which tapers from the uniform outer diameter to a smaller outer diameter at the proximal end 222 of mandrel 212. The tapering portion 233B allows the proximal end of the wire guide to have more flexibility in order to distribute stress caused by the flexibility of the wire guide. Proximal portion 233B of mandrel 212 tapers from the uniform outer diameter to a smaller outer diameter at a rate of about 0.0005 inches/1-inch span to about 0.004 inches/1-inch span. The annular space 215 defined between the tapering proximal portion 233B and the interior surface 211 of the lumen of cannula 214 can be filled with a filler material 225, such as a polymeric material like a urethane or others described herein. Mandrel 212 having the tapered proximal portion described herein can also be attached to slotted cannula 114 for improved flexibility.

A wire guide can be made having one or more features described herein, or any combinations thereof, with respect to the figures. Further detail, however, will be given to a method of manufacturing a wire guide with the slotted cannula of FIG. 2. As appreciated by one of ordinary skill in the art, various embodiments of wire guides, as described herein, can be made with similar steps. Specific materials and sizes used in describing the method of making the wire guide are given for exemplary reasons, and other materials and sizes are within the scope of the invention.

A stainless steel cannula having an outer diameter of 0.018 inches and a wall thickness of 0.004 inches is cut to a specified length. The slots are laser cut to have the size, shape and/or pattern as described herein to form cannula 114. For example, each slot is laser cut to have a width of about 0.015 inches and to have a triangular shape with angle 155 of about 110 degrees between edges 152, 153 of the slot.

Each series of slots can be laser cut in consecutive order or concurrently. Cannula 114 includes a first series 164 of slots and a second series 166 of slots disposed along three discrete longitudinal segments 172A-C. A slot 168 of the first series 164 is disposed along a portion of a first circumferential region of cannula 114, and an adjacent slot 170 of the first series 164 is disposed along a portion of a second circumferential region of the cannula. Slot 168 of the first circumferential region and slot 170 of the second circumferential region are circumferentially disposed relative to one another at angle 174 of about 20 degrees. Slots of the second series 166 are shown interposed between adjacent slots of the first series 164. A second series slot 169 of a first circumferential region, interposed between slots 168, 170 of the first series 164, and the second series slot 171 of a second, adjacent circumferential region are circumferentially disposed relative to one another at an angle, identical to angle 174, of also about 20 degrees. The slot 168 of the first series 164 and the slot 169 of the second series 166 are shown circumferentially disposed relative to one another at an angle 183 of about 90 degrees.

The slots of each series within first longitudinal segment 172A are longitudinally spaced from one another at a distance of about 0.016 inches, while the distance between a first series slot and an adjacent second series slot is about 50% of the longitudinal distance between slots of the same series, or about 0.008 inches. The slots of each series within second longitudinal segment 172B have an increased longitudinally spacing from one another of a distance of about two times the distance, or 0.032 inches, of the slots in first longitudinal segment 172A, while the distance between a first series slot and a second series slot is about 0.016 inches. The slots of each series within third longitudinal segment 172C have an even larger spacing from one another of a distance of about four times the distance, or 0.064 inches, of the slots in first longitudinal segment 172A, while the distance between a first series slot and a second series slot is about 0.032 inches. The slots end at longitudinal segment 172C and a portion 172D of cannula 114 from the end of the last slot to the proximal end 124 of the cannula does not have any slots. The most distal slot of the first longitudinal segment 172A is spaced from the distal end 128 of cannula 114 by a distance of 1 inch, although it can be any distance. The longitudinal distance of the first longitudinal segment 172A is about 5 inches, of the second longitudinal segment is about 1 inch, and of the third longitudinal segment is about 1 inch.

With reference to FIG. 1, a Nitinol wire having an outer diameter of 0.01 inches is cut to a specified length to form mandrel 12. The wire is ground and/or polished at the distal end to form a taper, preferably continuously smooth taper, from the uniform outer diameter to an outer diameter of about 0.005 inches at the distal end for a span of about 3 inches. If desired, the proximal end of the wire can be ground and/or polished to form a continuously smooth taper, such as shown in FIG. 4.

The tapered mandrel 12 is then inserted through the lumen of the slotted cannula 114, and axially positioned such that the proximal ends of the respective cannula and the mandrel are aligned and the tapered portion of the mandrel extends past the distal end of the cannula, as shown in FIG. 1. The mandrel and the cannula are attached by soldering the proximal ends together to form first attachment 20 and by soldering the distal end of the cannula to an intermediate portion of the mandrel to form second attachment 26 to form the wire guide. The solder at the second attachment is preferably circumferentially around the mandrel adjacent the distal end of the cannula. The second attachment solder is then ground and/or polished to form a smooth transition from the outer diameter of the cannula to the outer diameter of the mandrel. A second coating 44 of Teflon may be applied to the cannula as described herein. The second coating may then be cut and/or ground to size. A first coating 42 may be applied to the distal tip by dipping the distal tip into a urethane polymer. The first coating may also be cut and/or ground to size such that the difference between the outer diameter formed by the Teflon coated cannula and the polymer coated distal tip is less than 0.005 inches. A third hydrophilic coating may then be applied to the distal tip along the exterior surface of the first coating by a dipping process. The third coating may also be cut and/or ground to size such that the difference between the outer diameter formed by the Teflon coated cannula and the coated distal tip is negligible.

Although the invention has been described and illustrated with reference to specific illustrative embodiments thereof, it is not intended that the invention be limited to those illustrative embodiments. Those skilled in the art will recognize that variations and modifications can be made without departing from the true scope and spirit of the invention as defined by the claims that follow. It is therefore intended to include within the invention all such variations and modifications as fall within the scope of the appended claims and equivalents thereof.

We claim:

1. A wire guide for use in a body vessel, comprising:
an elongated mandrel having a proximal end and a distal end, the mandrel having a stiffness; and
a cannula having a proximal end and a distal end, and a lumen extending therebetween, the lumen sized to receive the mandrel therein, the cannula having a stiffness greater than the mandrel stiffness, and having a length relative to a length of said mandrel such that a portion of said mandrel extends distal to said cannula when received in said cannula lumen, the cannula being attached along the length of the mandrel at least at one attachment joint,
wherein the extended portion of the mandrel has a taper from a first diameter to a second diameter to form a distal tip of said wire guide.

2. The wire guide of claim 1, wherein the elongated mandrel is a shape memory alloy.

3. The wire guide of claim 1, wherein the cannula is selected from the group consisting of a stainless steel alloy, platinum alloy, palladium alloy, a nickel-titanium alloy and combinations thereof.

4. The wire guide of claim 1, further comprising a polymer coating surrounding the extended portion of the mandrel to form the distal tip, wherein at least a portion of the polymer coating has a hydrophilic coating disposed thereon for lubricity.

5. The wire guide of claim 4, further comprising a lubricious polymer coating disposed on at least one of a portion of the cannula or a portion of the extended portion of the wire guide.

6. The wire guide of claim 1, wherein the proximal end of the cannula is attached to the proximal end of the mandrel to form a first attachment joint, and the distal end of the cannula is attached to an intermediate portion of the mandrel, in between the proximal and distal ends thereof, to form a second attachment joint.

7. The wire guide of claim 1, wherein the mandrel further comprises a proximal taper from an intermediate portion to the proximal end to vary the stiffness of the mandrel.

8. The wire guide of claim 1, wherein the cannula further comprises a plurality of slots formed in a wall of the cannula, the slots being circumferentially disposed along the cannula wall.

9. The wire guide of claim 8, wherein the mandrel is disposed about a longitudinal axis and said slot is formed in the cannula wall transverse to said longitudinal axis.

10. The wire guide of claim 8, wherein the plurality of slots comprises a first slot and a second slot, where the first slot is disposed along a first portion of a circumferential region, and the second slot is disposed along a second portion of the same circumferential region of the first slot.

11. The wire guide of claim 8, wherein the plurality of slots comprises at least one series of slots, the slot of a series being spaced from an adjacent slot of the series at a longitudinal distance, wherein said longitudinal distance at a proximal portion of the cannula is greater than the longitudinal distance at a distal portion of the cannula.

12. The wire guide of claim 11, wherein the slots of the series are circumferentially positioned relative to one another at an angle to form a generally helical pattern along the cannula wall.

13. The wire guide of claim 11, wherein the series of slots is a first series, and the plurality of slots further comprises a second series of slots, each slot of the second series being interposed between adjacent first series slots.

14. The wire guide of claim 13, wherein each of the second series slots are circumferentially positioned relative to each of the first series slots at an angle such that the second series slots are circumferentially offset from the first series.

15. A wire guide for use in a body vessel, comprising:
an elongated mandrel having a proximal end and a distal end, the mandrel having a stiffness; and
a cannula having a proximal end and a distal end, and a lumen extending therebetween, the lumen sized to receive the mandrel therein, the cannula having a stiffness greater than the mandrel stiffness, and having a length relative to a length of the said mandrel such that a portion of said mandrel extends distal to said cannula when received in said cannula lumen,
the cannula comprising a plurality of longitudinally adjacent slots formed in a wall of the cannula, said longitudinally adjacent slots being circumferentially disposed along said cannula wall such that said longitudinally adjacent slots form a generally helical pattern along the cannula wall,
wherein the extended portion of the mandrel has a taper from a first diameter to a second diameter to form a distal tip of said wire guide.

16. The wire guide of claim 15, wherein the mandrel is disposed about a longitudinal axis and each of said longitudinally adjacent slots is formed in the cannula wall transverse to said longitudinal axis.

17. The wire guide of claim 16, wherein the plurality of longitudinally adjacent slots comprises at least one series of slots, the slot of a series being spaced from an adjacent slot of the series at a longitudinal distance, wherein said longitudinal distance at a proximal portion of the cannula is greater than the longitudinal distance at a distal portion of the cannula.

18. The wire guide of claim 17, wherein the series of slots is a first series, and the plurality of slots further comprises a second series of slots, each slot of the second series being interposed between adjacent first series slots, said longitudinally adjacent second series slots being circumferentially disposed along said cannula wall such that said longitudinally adjacent second series slots form a generally helical pattern along the cannula wall.

19. A method of making a wire guide for use within a body vessel, the method comprising:
providing an elongated mandrel having a proximal end and a distal end, the mandrel having a stiffness, wherein a portion of the mandrel has a taper from a first diameter to a second diameter;
providing a cannula having a proximal end and a distal end, and a lumen extending therebetween, the lumen sized to receive the mandrel therein, the cannula having a stiffness greater than the mandrel stiffness;
inserting the elongated mandrel into the cannula lumen such that a portion of said mandrel extends distal to said cannula; and
attaching a portion of the cannula to the mandrel to form a wire guide.

20. The method of claim 19, further comprising forming a plurality of slots in a wall of the cannula, each slot being circumferentially disposed along the cannula wall.
